(19) United States
(12) Patent Application Publication
(10) Pub. No.: US 2024/0400604 A1
(43) Pub. Date: Dec. 5, 2024

(54) NUCLEOSIDE TRIPHOSPHATE PHOTOAFFINITY PROBE, METHOD FOR PREPARING SAME, AND APPLICATIONS THEREOF

(71) Applicant: SHANDONG UNIVERSITY, Jinan (CN)

(72) Inventors: Rong CAI, Jinan (CN); Can GAO, Jinan (CN); Mengxuan LI, Jinan (CN); Zhiming WANG, Jinan (CN); Jing TAN, Jinan (CN); Wenwen LI, Jinan (CN); Jing XU, Jinan (CN)

(73) Assignee: SHANDONG UNIVERSITY, Jinan (CN)

(21) Appl. No.: 18/732,149

(22) Filed: Jun. 3, 2024

(30) Foreign Application Priority Data
Jun. 2, 2023 (CN) 2023106471801

(51) Int. Cl. C07H 19/20; C12Q 1/48; G01N 30/02; G01N 30/72; G01N 30/88; G01N 33/58

(52) U.S. Cl. CPC C07H 19/20; C12Q 1/485; G01N 30/7233; G01N 30/88; G01N 33/58; G01N 2030/027; G01N 2030/8831

(57) ABSTRACT

A nucleoside triphosphate photoaffinity probe, method for preparing same, and applications thereof. The nucleoside triphosphate photoaffinity probe is a novel small molecule activity probe for detecting nucleoside triphosphate-binding proteins and is based on the structure of nucleoside triphosphates (GTP and ATP) connected to a smaller photoaffinity side chain modification. This probe can effectively label nucleoside triphosphate-binding proteins in cell lysates for high-throughput proteomics analysis, identify and analyze the binding sites of the probe, and can also be used to analyze the action sites of nucleoside triphosphate competitive inhibitors, thus having significant practical application value.

### TECHNICAL FIELD

The present invention belongs to the technical field of biological detection and analysis, specifically relating to a nucleoside triphosphate photoaffinity probe, method for preparing same, and applications thereof.

### BACKGROUND

The information disclosed in this section of the background technology is merely intended to enhance the overall understanding of the background of the invention and should not necessarily be taken as an acknowledgment or any form of implication that this information constitutes prior art that is already known to those skilled in the art.

Nucleoside triphosphate are crucial metabolites within cells. For example, adenosine triphosphate (ATP) serves as the major energy carrier in living organisms, and guanosine triphosphate (GTP) acts as an important signaling molecule. Their key roles in cellular activities are mainly realized through proteins with which they interact. GTP-binding proteins play important roles in many fundamental cellular processes, including signal transduction, intracellular transport, cell proliferation, differentiation, and apoptosis. G proteins are a class of GTP-binding proteins that act as molecular switches in the process of cell signaling. Mutations or dysregulation of their activities usually associated with disease development, such as persistently activated small G protein KRAS mutants, frequently detected in various malignant tumors. G proteins are involved in multiple signaling pathways, regulated upstream by receptors like the epidermal growth factor receptor and G protein-coupled receptors, and participating in downstream pathways such as the cAMP signaling pathway and the MAPK pathway, impacting gene expression, transcription, and cell proliferation. Considering the pivotal role of G proteins, analyzing their activity and mechanism is of great importance.

Currently, there are two primary methods for detecting G proteins activities. The first involves using effector proteins that selectively bind to the active state of G proteins for immunoprecipitation and protein immunoblotting analyses. The other estimates G protein enzymatic activity by quantifying the free phosphate molecules produced during GTP hydrolysis. The first method is mainly suitable for determining the activity of specific G proteins, requiring different proteins and antibodies for analysis of different G proteins, which is costly and cannot analyze multiple different G proteins simultaneously. The second high-throughput method can assess G protein enzymatic activity under different conditions or drug treatments, but cannot distinguish which G protein corresponds to the detected activity from a protein mixture.

ATP-binding proteins are involved in various cellular processes, including cell metabolism, cell signaling, biosynthesis, transport, migration, etc. Since ATP contains high-energy phosphate bonds that can be hydrolyzed to generate ADP by phosphatases while transferring energy to cells. Many ATP-binding proteins require ATP hydrolysis as an energy source. Among these proteins, kinases are key regulators in several cell signaling processes and aberrant kinase signaling contributes to the development of many human diseases, including cancer. Kinases could transfer the γ-phosphate group from ATP to specific amino acids of substrate proteins, thereby affecting the structure and activity of the substrate protein, and participating in a series of cell signal transduction and regulation processes. Due to the association of abnormal kinase activities with diseases, kinases are critical drug targets. Most kinase inhibitors target the ATP-binding pockets to compete with ATP binding and inhibit kinase activities. Considering the conserved ATP-binding pockets among ATP-binding proteins, developing high-throughput analytical methods to evaluate the efficacy and selectivity of various kinase inhibitors is of great importance.

Currently reported kinase inhibitor probes are mainly based on two types of core structures. The first type is based on kinase inhibitor analogs, such as the Kinobeads technology, which uses affinity resins linked with multiple kinase inhibitors to broadly enrich various kinases. In this process, the test compound is added to competitively bind target kinases, reducing the Kinobeads enrichment of kinases targeted by the test compound, followed by quantitative analysis through liquid chromatography-mass spectrometry (LC-MS) to identify potential target proteins of the test compound. In addition, this type of probes can also be used to analyze the target proteins of specific kinase inhibitors. The second type is based on ATP analogs. Unlike kinase inhibitors that have a certain selectivity towards different kinases, ATP can bind with almost all kinases. Compared with probes designed based on kinase inhibitors, ATP analog probes can identify a more diverse range of kinases and analyze different ATP-binding proteins. For example, in the commonly used Kinativ technology, biotinylated ATP acyl-phosphate probes could covalently react with conserved lysine residues in the ATP-binding pockets of kinases. Subsequently, ATP-binding proteins are enriched through the interaction between biotin and streptavidin. This probe has been employed for kinome profiling and evaluation of kinase inhibitors based on competitive labeling experiments. Although these probes are effective in labeling and identifying ATP-binding sites, their high reactivity results in poor stability and labeling selectivity, which needs to be further improved.

### SUMMARY

To address the deficiencies of the above-mentioned prior art, the present invention provides a nucleoside triphosphate photoaffinity probe, method for preparing same, and applications thereof. Based on the structure of nucleoside triphosphates, the invention connects a small photoaffinity side chain modification, developing a new small molecule active probe for detecting nucleoside triphosphate-binding proteins. The probe can effectively label nucleoside triphosphate-binding proteins in cell lysates for high-throughput proteomic analysis, identify and analyze the probe's binding sites, and analyze the action sites of nucleoside triphosphate competitive inhibitors. Based on the above research results, this invention was completed.

Specifically, the present invention involves the following technical solutions:
the first aspect of the invention provides a nucleoside triphosphate photoaffinity probe, having the structural formula as shown in formula I:

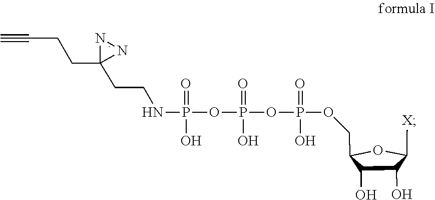

wherein X is selected from

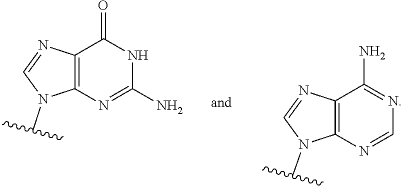 [SMILES: *n1cnc2c(=O)[nH]c(N)nc21 ; *n1cnc2c(N)ncnc21]

In the present invention, when X is

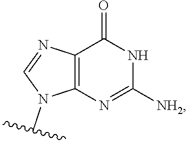 [SMILES: *n1cnc2c(=O)[nH]c(N)nc21]

the nucleoside triphosphate photoaffinity probe is named GTP-N probe, which can be used to identify GTP-binding proteins.

When X is

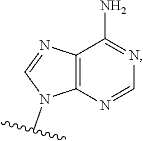 [SMILES: *n1cnc2c(N)ncnc21]

the nucleoside triphosphate photoaffinity probe is named ATP-N probe, which can be used to identify ATP-binding proteins.

The designed photoaffinity probes for nucleoside triphosphates contain binding groups GTP or ATP (abbreviated as GTP/ATP), photo-reactive groups diazirine, and bioorthogonal groups alkynes. The photo-reactive groups can form carbenes under UV irradiation, inserting into N—H, O—H, C—H bonds of amino acids, forming covalent bonds. Alkynes can undergo click chemistry reactions with azide compounds, linking fluorescent groups (e.g., rhodamine) or biotin groups (e.g., biotin-DADPS-azide, CAS: 1260247-50-4) for fluorescent imaging or affinity enrichment, and so on. Additionally, through acid-cleavable phosphoramidate bonds connecting GTP/ATP to modification groups, the large molecular weight GTP/ATP part can be dissociated after protein labeling, reducing molecular weight changes brought by probe modification, facilitating mass spectrometry analysis of modification sites. Experiments of the present invention verified that the above photoaffinity probes for nucleoside triphosphates can covalently capture GTP/ATP-binding proteins for subsequent analysis.

The second aspect of the present invention provides a method for the preparing the above nucleoside triphosphate photoaffinity probe, including the following synthetic route:

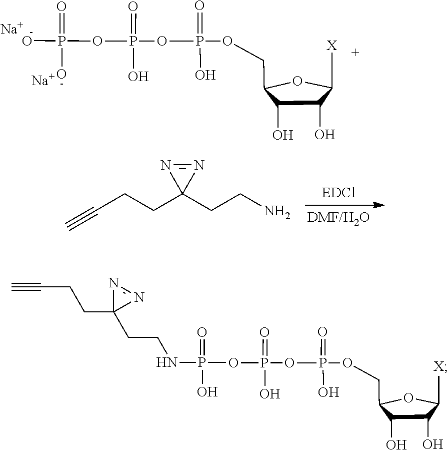

wherein X is selected from

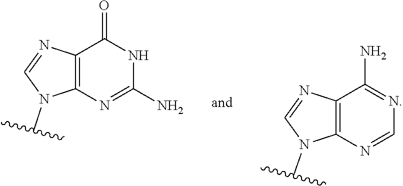 [SMILES: *n1cnc2c(=O)[nH]c(N)nc21 ; *n1cnc2c(N)ncnc21]

The third aspect of the present invention provides a detection kit, which at least contains the above-mentioned nucleoside triphosphate photoaffinity probe. Preferably, the detection kit may also contain other reagents (e.g., reaction enhancers, enzyme reagents, buffers, cleaning solutions).

In a specific embodiment of the present invention, the reagent is magnesium chloride, which can effectively enhance probe labeling at low concentrations (0.05-2.5 mM).

The fourth aspect of the present invention provides an application of the above nucleoside triphosphate photoaffinity probe or detection kit in GTP-binding protein-related research or ATP-binding protein-related research (abbreviated as GTP/ATP-binding protein-related research).

Specifically, the GTP/ATP-binding protein-related research at least includes:
(a) proteomic MS analysis of GTP/ATP-binding proteins in cell lysates; and
(b) analysis of G-protein inhibitors/kinase inhibitors.

In the application (a), a specific method includes: adding the nucleoside triphosphate photoaffinity probe to HEK 293T cell lysates, followed by UV irradiation, and performing click chemistry with biotin-DADPS-azide; specifically, using LC-MS/MS to detect and analyze the proteins labeled by the nucleoside triphosphate photoaffinity probe, assigning peptides from the obtained mass spectrometry data using MaxQuant software or Proteome Discoverer 2.5, and performing GO analysis using the DAVID website.

In the application (b), the G-protein inhibitor is EHT 1864, and the kinase inhibitor is staurosporine.

Specifically, the application (b) involves the analysis and identification of potential target proteins of G-protein inhibitors/kinase inhibitors.

Beneficial technical effects of the above one or more technical solutions:
the above technical solutions are based on the structure of GTP/ATP connected to a smaller photoaffinity side chain modification, developing a novel small molecule active probe for detecting GTP/ATP-binding proteins. The probe has three main advantages: firstly, by using a minimized linkage group, the modification's impact on the binding between GTP/ATP and proteins is reduced, better simulating the natural binding of GTP/ATP to proteins. Secondly, the probe contains an alkyne group for bioorthogonal reactions, allowing it to undergo click chemistry with azide compounds. This enables the linkage to fluorescent groups such as rhodamine for fluorescent gel imaging or biotin groups for affinity enrichment and mass spectrometry detection. Lastly, the probe connects GTP/ATP to the modification group via an acid-labile phosphoramidate bond, allowing the large molecular weight GTP/ATP portion of the probe to dissociate after protein labeling. This reduces the molecular weight change caused by the probe modification, facilitating the analysis of modification sites by mass spectrometry.

In summary, the probe can effectively label GTP/ATP-binding proteins in cell lysates for fluorescent imaging analysis, high-throughput proteomics analysis, and identification and analysis of the probe's binding sites. It can also be used to analyze the action sites of GTP/ATP competitive inhibitors, making it highly valuable for practical applications.

### BRIEF DESCRIPTION OF THE DRAWINGS

The drawings forming part of the specification are used to provide a further understanding of the present invention. The illustrative embodiments and their descriptions serve to explain the present invention and do not constitute undue limitations on the present invention.

FIG. 1: The structures of the probes; (a) GTP-O probe; (b) GTP-N probe; (c) GTP-ctrl probe.

FIG. 2: The design of the probes; (a) both the GTP-O probe and the GTP-N probe contain a binding group GTP, a photo-reactive group diazirine, and a bioorthogonal group alkyne; (b) click reaction between alkyne and azide to link the rhodamine fluorophore or biotin group; (c) based on the photocrosslinking reaction, the photo-cross linker can form a carbene under UV irradiation, inserting into the N—H, O—H, and C—H bonds of amino acids.

FIG. 3: Design of the GTP-N probe acid-cleavable group.

FIG. 4: Full MS spectra of the probe in negative ion mode.

FIG. 5: Tandem MS spectrum of the probe in negative ion mode.

FIG. 6: Stability analysis of the GTP-O probe under three pH conditions with HPLC.

FIG. 7: Stability analysis of the GTP-N probe under three pH conditions with HPLC.

FIG. 8: Full MS spectra for the GTP-ctrl probe treated with 1% TFA for different times.

FIG. 9: Analysis of GTP-N probe labeling efficiency under different labeling conditions; (a) different probe concentrations; (b) different incubation time; (c) different UV irradiation times; (d) different click reaction time. Unless otherwise specified in the figure, 50 μM probe, 10-min incubation time, 30-min UV irradiation time, and 2-hour click reaction time were used.

FIG. 10: Comparison between labeling efficiencies of different probes; (a) labeling using 0.1 mM GTP-O probe or GTP-N probe with or without 1 mM GTP; (b) labeling using GTP-N probe and GTP-ctrl probe under different concentrations. Unless otherwise specified in the figure, 1 mM MgCl₂, 10-min incubation time, 30-min UV irradiation time, and 2-hour click reaction time were used.

FIG. 11: Effect of different competitors on the labeling efficiency of the GTP-O probe; (a) competition effect of GTP or ATP; (b) competition effect of GTP, GDP and GMP. Unless otherwise specified in the figure, 50 μM probe, 1 mM MgCl₂, 10-min incubation time, 30-min UV irradiation time, and 2-hour click reaction time were used.

FIG. 12: Proteomic analysis of enriched proteins after GTP-O probe labeling; (a) Venn diagram showing the total proteins identified in three biological replicates using the GTP-O probe; (b) Venn diagrams showing the GTP-binding proteins identified in three biological replicates using the GTP-O probe.

FIG. 13: Proteomic analysis of enriched proteins after GTP-N probe labeling; (a) Venn diagram showing the total proteins identified in three biological replicates using the GTP-N probe; (b) Venn diagram showing the GTP-binding proteins identified in three biological replicates using the GTP-N probe.

FIG. 14: Comparison of identified protein number and intensity between the GTP-N probe and the GTP-ctrl probe; (a) Venn diagram showing the comparison of all identified proteins; (b) Venn diagram showing the comparison of GTP-binding proteins; (c) comparison of the MS intensity of commonly identified proteins using the GTP-N probe or the GTP-ctrl probe.

FIG. 15: LFQ analysis of the competitive effect of GTP on the GTP-O probe or GTP-N probe labeling; (a) Volcano plot showing the quantification results for three biological replicates with GTP-O probe labeling compared with competitive labeling with GTP; (b) Volcano plot showing the quantification results for three biological replicates with GTP-N probe labeling compared with competitive labeling with GTP; (c) GO-MF analysis of GTP-O probe labeled proteins with ratio<1; (d) GO-MF analysis of GTP-N probe labeled proteins with ratio<1.

FIG. 16: Representative tandem MS spectrum of a probe-labeled peptide from GTP-binding protein EEF1A1.

FIG. 17: Heatmap showing the changes of protein intensities after competitive probe labeling with EHT 1864; (a) changes in intensity of all identified proteins after competitive probe labeling with EHT 1864; (b) changes in intensity of GTP-binding protein after competitive probe labeling with EHT 1864; (c) changes in intensity of EHT 1864-related protein; (d) changes in intensity of proteins with a decreasing trend after competitive probe labeling with EHT 1864. Using the intensity ratio (inhibitor competition group/no inhibitor probe group) plotted with log₂ values; probe concentration was 20 PM, and EHT 1864 concentrations was 1×, 2×, 5×, and 10× of the probe concentration.

FIG. 18: Full MS spectra of the ATP probes in negative ion mode; (a) ATP-ctrl probe; (b) ATP-N probe.

FIG. 19: ¹H-NMR spectrum of the ATP-ctrl probe.

FIG. 20: ³¹P-NMR spectrum of the ATP-ctrl probe.

FIG. 21: ¹H-NMR spectrum of the ATP-N probe.

FIG. 22: ³¹P-NMR spectrum of the ATP-N probe.

FIG. 23: Analysis of ATP-N probe labeling under different conditions. (a) different probe concentrations; (b) different probes with or without UV irradiation; (c) effect of different competitors. Unless otherwise specified in the figure, 10 μM probe, 10-min incubation time, 30-min UV irradiation time, and 2-hour click reaction time were used.

FIG. 24: LFQ analysis of the competitive effect of ATP on the ATP-N probe labeling; (a) Volcano plot showing the quantification results for three biological replicates with ATP-N probe labeling compared with competitive labeling with ATP; (b) GO-MF analysis of labeled proteins with normalized abundance ratio>2 and p-value<0.05.

FIG. 25: LFQ analysis of the competitive effect of ATP on ATP-N probe labeled peptides; (a) Volcano plots showing the quantification results for three biological replicates with ATP-N probe labeling compared with competitive labeling with ATP for labeled peptides; (b) GO-MF analysis of labeled peptides with normalized abundance ratio>2 and p-value<0.05.

FIG. 26: Identified probe-labeled peptides for representative kinases; (a) representative structure and tandem MS spectrum of probe-labeled peptides of NAGK; (b) representative structure and tandem MS spectrum of probe-labeled peptides of VRK1.

FIG. 27: Kinases that showed decreased probe-labeling efficiency with increasing staurosporine concentration; a heatmap showing kinases with decreased MS signal intensity after competitive probe labeling with staurosporine at 1×, 10×, and 20× probe concentration, highlighting significantly decreased kinases.

### DETAILED DESCRIPTION

It should be noted that the terminology used herein is for the purpose of describing particular embodiments only and is not intended to be limiting of the exemplary embodiments of the present invention. As used herein, unless the context clearly indicates otherwise, the singular forms are intended to include the plural forms as well. Moreover, it should be understood that when the terms “comprise” and/or “include” are used in this specification, they specify the presence of stated features, steps, operations, devices, components, and/or combinations thereof. If the experimental methods in the following specific embodiments do not specify particular conditions, they are generally conducted according to conventional methods and conditions well-known in the art, which are fully described in the literature.

The following specific examples are provided to further illustrate the present invention and are not intended to limit the content of the present invention. If the specific conditions of the experiments are not specified in the examples, they are generally carried out under conventional conditions or according to the conditions recommended by the suppliers. Unless otherwise stated, the materials, reagents, and the like used in the examples can be obtained commercially.

The following examples are provided to further illustrate the present invention but are not intended to limit the scope of the present invention. It should be understood that these examples are merely for the purpose of illustrating the present invention and are not intended to limit the scope of the present invention.

### Example 1 Design and Synthesis of Novel GTP Photoaffinity Probes

### 1. Materials and Methods

1.1 Experimental Reagents Methanol and acetonitrile (Oceanpak), N,N-Dimethylformamide (Adamas), dimethyl sulfoxide (Sigma-aldrich), tetrabutylammonium hydroxide (Sigma-aldrich), triethylamine (Energy), hydrochloric acid, acetic acid and sodium hydroxide (Sinopharm), pyridine (AcmecBiochemical), Dowex 50WX4 ion-exchange resin 100-200 (H) (Alfa), guanosine-5′-triphosphate disodium salt (Dibo), 3-(But-3-yn-1-yl)-3-(2-iodoethyl)-3H-diazirine, 2-(3-(But-3-yn-1-yl)-3H-diazirin-3-yl)ethan-1-amine, 5-hexylene-1-amine, 1-(3-dimethylaminopropyl)-3-ethylcarbodiimide hydrochloride, and sodium carbonate (Bide), sodium chloride (Adamas), trichloromethane (Tieta), trifluoroacetic acid (Energy), phosphate-buffered saline (PBS) buffer (Solarbio).

### 1.2 Experimental Equipment

Thermostatic magnetic stirrer (IKA), centrifuge (Eppendorf), HPLC (Agilent 1260), Mass spectrometer (Thermo Fisher LCQ Fleet), lyophilizer (Beijing Sihuan), UV spectrometer (Shimadzu), semi-preparative column (Welch, Ultimate AQ-C18, 5 μm, 10×250 mm), analytical column (Welch, Ultimate XB-C18, 5 μm, 4.6×250 mm), thermostatic metal bath (Mini H100), NMR (Bruker), pH meter (Sartorius).

### 1.3 Experimental Procedures

### 1.3.1 Synthesis and Purification of the Probes

### Gtp-O Probe:

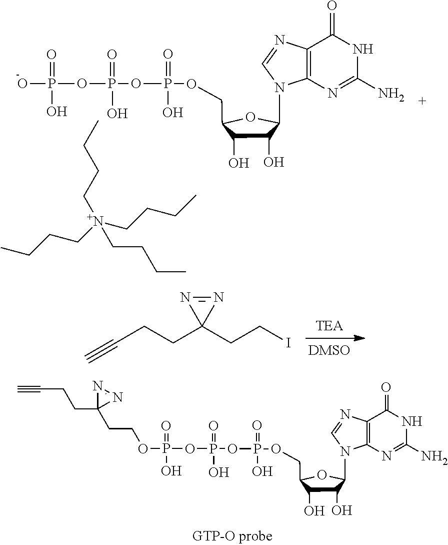

GTP tetrabutylammonium salt was prepared from GTP disodium salt by passing the aqueous solution through a cation-exchange column. The eluent was neutralized with tetrabutylammonium hydroxide and lyophilized to yield a white solid. In a round-bottom flask protected from light, GTP tetrabutylammonium salt (76 mg, 0.1 mmol) was dissolved in 0.64 mL DMSO and 3-(But-3-yn-1-yl)-3-(2-iodoethyl)-3H-diazirine (0.03 ml, 0.2 mmol) was added, followed by triethylamine (68 μL, 0.48 mmol). The mixture was stirred at room temperature for 52 hrs and dried. The residue was dissolved in water and purified using a C18 column (Welch, Ultimate AQ-C18, 5 μm, 10×250 mm). The mobile phase A was 50 mM tetraethylammonium acetate (TEAA) buffer (pH=8), the mobile phase B was 50 mM TEAA in 30% ACN, the flow rate was 2 mL/min, the detection wavelength was 254 nm, and the column temperature is 25° C. Gradient elution conditions were as follows: 0-5 minutes, 5% B; 5-10 minutes, 5-30% B; 10-46 minutes, 30-48% B; 46-51 minutes, 48-100% B; 51-61 minutes, 100% B; 61-66 minutes, 100-5% B; 66-71 minutes, 5% B. The GTP-O probe eluted at 43-45 minutes, and the fractions containing the pure product were collected, freeze-dried, yielding a white solid. The probe concentration was determined by its UV absorption at 252 nm (extinction coefficient ε=13700 M⁻¹ cm⁻¹), and the yield was 16.7%. The probe was aliquoted and stored at −80° C. The structure and purity of the probe were confirmed with MS, HPLC and NMR. ¹H NMR (400 MHz, D₂O): δ 8.11 (s, 1H), 5.91 (d, J=6.1 Hz, 1H), 4.51 (dd, J=5.2, 3.3 Hz, 1H), 4.35-4.29 (m, 1H), 4.21 (dd, J=5.5, 3.4 Hz, 2H), 3.77 (qd, J=6.6, 2.2 Hz, 2H), 2.29 (t, J=2.7 Hz, 1H), 1.94 (td, J=7.3, 2.6 Hz, 2H), 1.88 (s, 2H), 1.65 (td, J=6.3, 2.7 Hz, 2H), 1.59 (t, J=7.2 Hz, 2H). ³¹P NMR (162 MHz, D₂O): δ-11.43 (dd, J=17.8, 9.2 Hz, 2P), −22.70 (t, J=16.2 Hz, 1P).

### Gtp-N Probe:

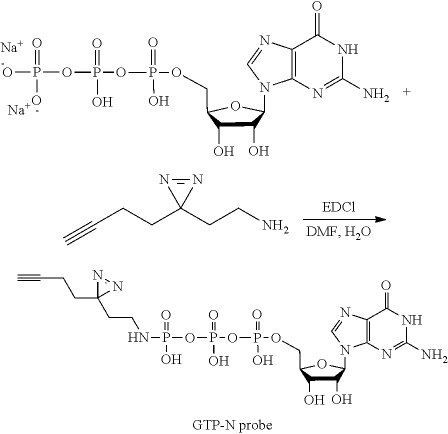

The GTP disodium salt (56.7 mg, 0.1 mmol) was dissolved in 1 mL H₂O followed by the addition of a solution of EDCI (95.8 mg, 0.5 mmol) in 1 mL H₂O. The mixture was stirred at 25° C. in the dark for 7 min. Then, 2-(3-(But-3-yn-1-yl)-3H-diazirin-3-yl)ethan-1-amine (14.11 L, 0.11 mmol) was dissolved in 2 mL DMF, added to the reaction flask and stirred at 25° C. for 4.5h. The reaction mixture was extracted three times with 2 mL of chloroform, the aqueous phase was transferred to a centrifuge tube. Sodium chloride (0.2455 g) and 30 mL of anhydrous ethanol were added, followed by overnight precipitation at −20° C. After centrifugation at 4° C. for 10 minutes at 4200 rpm, the supernatant was discarded, and the resulting white solid was dissolved in a small amount of water and purified using HPLC under conditions similar to those used for the GTP-O probe. Gradient elution conditions were as follows: 0-5 minutes, 5% B; 5-10 minutes, 5-30% B; 10-37 minutes, 30-34.5% B; 37-42 minutes, 34.5-100% B; 42-47 minutes, 100% B; 47-52 minutes, 100-5% B; 52-57 minutes, 5% B. The GTP-N probe eluted at 28-30 minutes, probe-containing eluents were combined and lyophilized to yield a white solid. The probe concentration was determined by UV absorption at 252 nm (extinction coefficient ε=13700 M⁻¹ cm⁻¹), and the yield was 32.3%. The probe was aliquoted and stored at −80° C. The structure and purity of the probe were confirmed with MS, HPLC and NMR. ¹H NMR (400 MHz, D₂O): δ 8.11 (s, 1H), 5.90 (d, J=6.2 Hz, 1H), 4.52 (dd, J=5.2, 3.3 Hz, 1H), 4.32 (dt, J=5.4, 2.6 Hz, 1H), 4.21 (dd, J=5.6, 3.5 Hz, 2H), 2.75-2.62 (m, 2H), 2.30 (t, J=2.6 Hz, 1H), 1.93 (td, J=7.3, 2.7 Hz, 2H), 1.90 (s, 3H), 1.60-1.53 (m, 2H), 1.50 (t, J=7.3 Hz, 2H). ³¹P NMR (162 MHz, D₂O): δ-1.72 (d, J=20.5 Hz, 1P), −11.49 (d, J=19.4 Hz, 1P), −22.81 (t, J=19.8 Hz, 1P).

### GTP-Ctrl Probe:

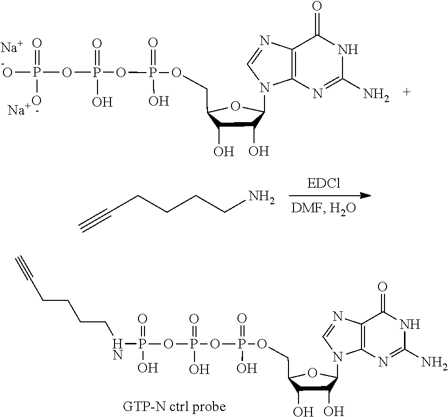

The GTP disodium salt (113.4 mg, 0.2 mmol) was dissolved in 2 mL H₂O followed by the addition of a solution of EDCI (191.7 mg, 1.0 mmol) in 2 mL H₂O. The mixture was stirred at 25° C. in the dark for 7 min. Then, 5-hexylene-1-amine (21.4 mg, 0.22 mmol) was dissolved in 4 mL DMF and added to the reaction flask, followed by the same steps as the synthesis of the GTP-N probe. Gradient elution conditions were as follows: 0-5 minutes, 5% B; 5-20 minutes, 5-15% B; 20-45 minutes, 15-20% B; 45-51 minutes, 20-22% B; 51-56 minutes, 22-100% B; 56-66 minutes, 100% B; 66-71 minutes, 100-5% B; 71-76 minutes, 5% B. The GTP-ctrl probe eluted at 44-46 minutes, probe-containing eluents were combined and lyophilized to yield a white solid. The probe concentration was determined by UV absorption at 252 nm (extinction coefficient ε=13700 M⁻¹ cm⁻¹), and the yield was 9%. The probe was aliquoted and stored at −80° C. The structure and purity of the probe were confirmed with MS, HPLC and NMR. ¹H NMR (400 MHz, D₂O): δ 8.01 (s, 1H), 5.79 (d, J=6.2 Hz, 1H), 4.42 (dd, J=5.1, 3.3 Hz, 1H), 4.25-4.18 (m, 1H), 4.10 (t, J=4.5 Hz, 2H), 2.71 (q, J=7.3 Hz, 2H), 2.18-2.11 (m, 1H), 1.99 (td, J=6.9, 2.6 Hz, 2H), 1.79 (d, J=0.8 Hz, 3H), 1.35-1.26 (m, 4H). ³¹P NMR (162 MHz, D₂O): δ-1.07 (d, J=20.9 Hz, 1P), −11.57 (d, J=19.2 Hz, 1P), −22.98 (t, J=20.1 Hz, 1P).

### 1.3.2 Stability Analysis of the Probes

Three pH conditions were selected to test the stability of the GTP-O probe and the GTP-N probe: a 70% acetonitrile solution containing 1% trifluoroacetic acid (pH=3), PBS buffer (pH=7.4) and carbonate buffer (pH=12). Probes were incubated with 0.4 mM final concentration in different buffer at 37° C. for different time, and 10 μL of solution was taken out at each time point for HPLC analysis. A analytical column (Welch, Ultimate XB-C18, 5 μm, 4.6×250 mm) was used for the analysis, and untreated probes were used as controls. The mobile phase A was 50 mM tetraethylammonium acetate (TEAA) buffer (pH=8), the mobile phase B was 50 mM TEAA in 30% ACN, the flow rate was 1 mL/min, the detection wavelength was 252 nm, and the column temperature was 30° C. Gradient elution conditions were as follows: 0-5 minutes, 5% B; 5-10 minutes, 5-30% B; 10-30 minutes, 30-40% B; 30-35 minutes, 40-100% B; 35-40 minutes, 100% B; 45-50 minutes, 5% B. For the GTP-ctrl probe, a small amount of sample was taken out after incubating for 5 minutes and 30 minutes in a 1% trifluoroacetic acid solution for mass spectrometry analysis.

### 2. Results and Discussion

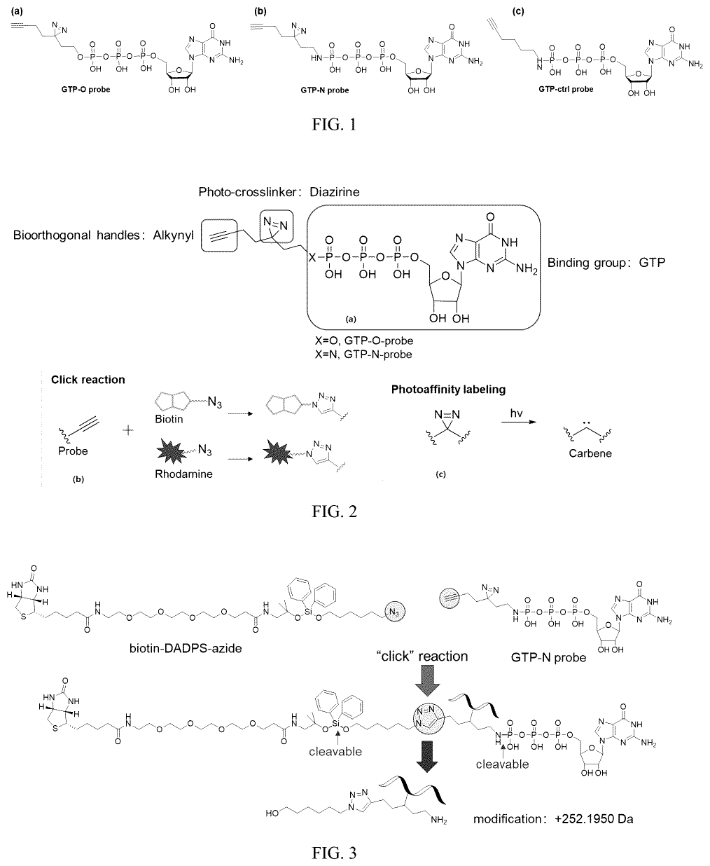

By connecting GTP with small modifying groups, three GTP probes were synthesized (FIG. 1). The GTP-O probe and the GTP-N probe contain binding group GTP, photoreactive group diazirine, and biorthogonal group alkyne; while the GTP control probe contains binding group GTP and biorthogonal group alkyne without photoreactive group. The photoreactive group can form a carbene under UV irradiation, inserting into the N—H, O—H, and C—H bonds of amino acids to form a covalent connection. The alkyne group can undergo click reaction with azides, linking fluorophores or biotin to labeled proteins for subsequent fluorescence imaging or affinity enrichment (FIG. 2). The control probe without the photoreactive group was mainly used to compare with the photoaffinity probes to analyze the influence of photoreactive groups on protein labeling.

In addition, a new GTP-N probe was designed based on the acid-labile phosphoramidate group that would break the linkage between GTP and the photoreactive diazirine group under acidic condition. After click reaction with the acid-cleavable biotin-DADPS-azide followed by acidic hydrolysis, both GTP and biotin could be released from the modification sites. This would reduce the molecular weight change caused by probe modification and decrease the interference by large modification groups on MS analysis (FIG. 3).

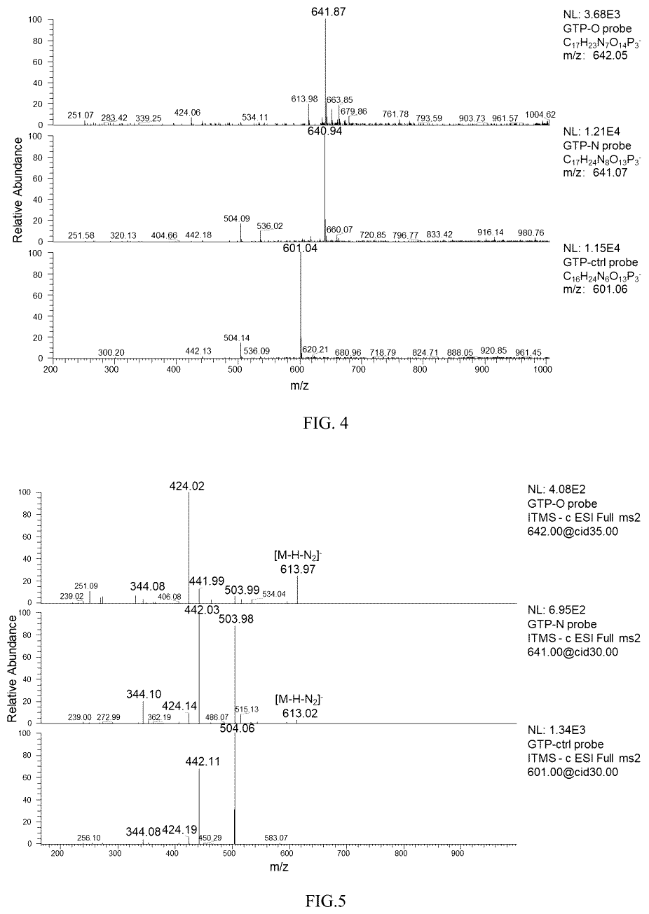

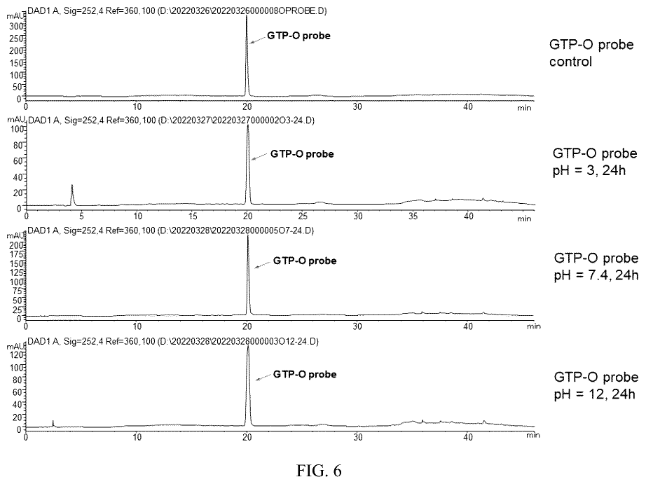

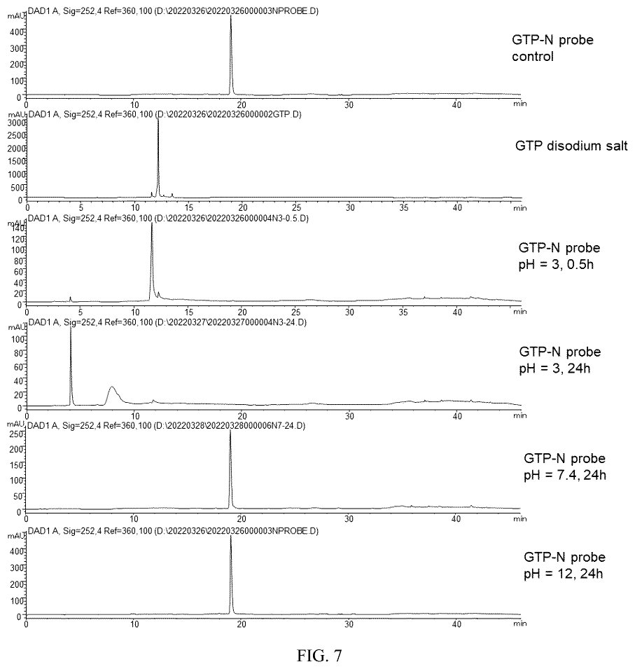

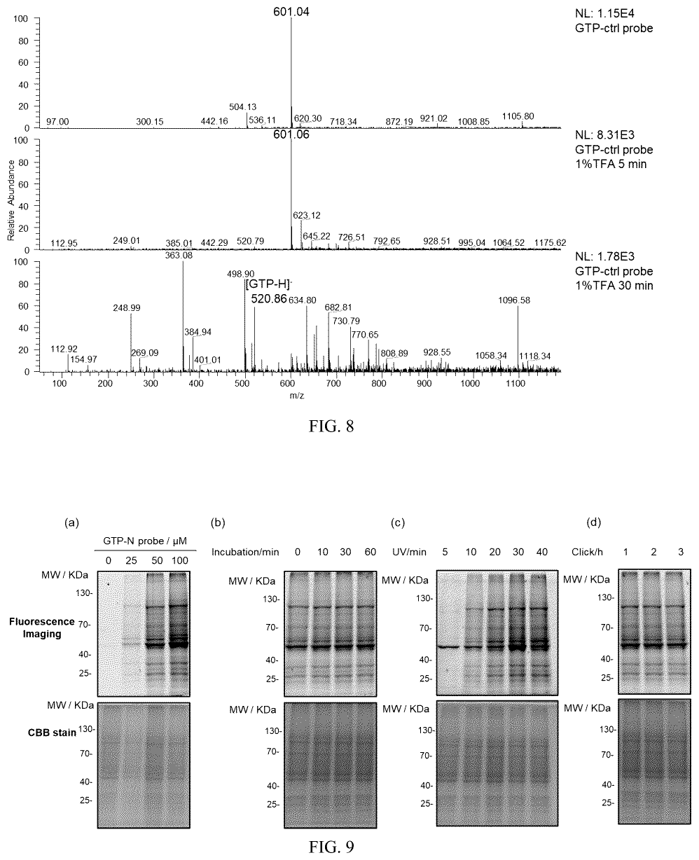

The three probes were characterized with mass spectrometry to confirm their structures (FIG. 4 and FIG. 5). In the stability analysis experiment, the GTP-O probe did not degrade within 24 hours under three pH conditions, indicating its good stability (FIG. 6). The GTP-N probe was stable for 24 hours under basic or neutral conditions, but decomposed under acidic condition with trifluoroacetic acid after 0.5 hours (FIG. 7). The control probe was incubated in 1% trifluoroacetic acid solution for different duration and tested with mass spectrometry to determine its dissociation products. When the control probe was incubated in 1% trifluoroacetic acid solution for 5 minutes, most of the probe remained unchanged. When incubated for 30 minutes, almost all of the probes decomposed into GTP (FIG. 8). This suggested the relatively good stability of this new type of GTP probes. Under acidic conditions, the GTP-N probe could dissociate and release GTP, reducing the molecular weight change caused by probe modification and facilitate subsequent MS-based binding site analysis.

### Example 2 in-Gel Fluorescence Imaging Analysis of GTP-Binding Proteins Labeled with Photoaffinity Probes in Cell Lysates

### 1. Materials and Methods

### 1.1 Experimental Reagents

Methanol, acetic acid and trolamine (Fuyu), guanosine-5′-triphosphate disodium salt, guanosine-5′-diphosphate disodium salt and guanosine 5′-monophosphate disodium salt (Dibo), sodium chloride (Titan), trichloromethane (Tieta), sodium bicarbonate, rhodamine β-PEG3-azide, tris(2-carboxyethyl)phosphine hydrochloride (TCEP), copper sulfate, tris[(1-benzyl-1H-1,2,3-triazol-4-yl)methyl]amine (TBTA), bromophenol blue and β-mercaptoethanol (Bide), 4-hydroxyethylpiperazine ethanesulfonic acid (HEPES), ethylenediaminetetraacetic acid (EDTA), sodium dodecyl sulfate (SDS), tris(hydroxymethyl)aminomethane hydrochloride (Tris-HCl) (all from Genview), Triton X-100 (Beyotime), magnesium chloride (Macklin), tetramethylethylenediamine (TEMED) and 30% Acr-Bis (Dingguo), Coomassie brilliant blue R-250, phosphatase inhibitor A, phosphatase inhibitor B and protease inhibitor (Abmole), Bradford protein assay kit and BCA protein pssay Kit (Beyotime), NAP-5 (Amersham).

### 1.2 Experimental Equipment

Pipettor and centrifuge (Eppendorf), thermostatic metal bath (Mini H100), microplate reader, electrophoresis apparatus (Bio-Rad), 365-nm ultraviolet lamp (ZF-1), Vortex (IKA), Horizontal shaker (DLAB), Gel imager (GE Amersham Imager 600), pH meter (Sartorius), NanoDrop one (Thermo Fisher).

### 1.3 Experimental Procedures

In-Gel Fluorescence Imaging of HEK 293T Cell Lysate Labeled with the Probes

Appropriate amount cells were incubated on ice for 30 min in 1% lysis buffer (1% Triton X-100, 50 mM HEPES (pH 7.4), 150 mM NaCl, supplemented with phosphatase inhibitor A, phosphatase inhibitor B, and protease inhibitor right before use) at 5-10 times the cell volume. Vortexing for 5 seconds every 10 minutes. The cell lysates were centrifuged at 12,000 rpm at 4° C. for 30 min, and the supernatants were passed through an Amersham NAP-5 column to remove endogenous nucleosides. The proteins were quantified using a Bradford protein assay kit. Approximately 100 μg protein was diluted with 0.1% lysis buffer (0.1% Triton X-100, 50 mM HEPES, 150 mM sodium chloride). To the solution was added 1 mM magnesium chloride, the GTP probe and competitors to designated concentrations. The final volume for the reaction was 100 μL and the final protein concentration was 1 μg/L. After incubated on ice, the solution was irradiated with 365-nm UV light for 30 min. To the solution were added freshly prepared tris-(2-carboxyethyl)phosphine (TCEP) until its concentration reached 1 mM, and a CuAAC reaction mixture to reach a final concentration of 1 mM CuSO₄, 0.1 mM tris[(1-benzyl-1H-1,2,3-triazol-4-yl)methyl]amine (TBTA), and 0.1 mM rhodamine β-PEG3-azide. The mixture was incubated at room temperature for 2 hrs and then precipitated in a solution of chloroform: methanol: water (4:3:1, v/v). After washing with cold methanol and air dried, the protein pellet was dissolved in 30 μL 4% SDS buffer (4% SDS, 50 mM HEPES (pH 7.4), 150 mM NaCl, 50 mM Trolamine). The protein concentration was determined using a BCA protein assay kit. A solution of approximately 20 μg proteins was added 4×loading buffer (0.25M Trometamol, pH=6.8, 4% SDS, 40% Glycerol, 0.02% bromophenol blue, add 200 μL 2-Mercaptoethanol to 800 μL buffer right before use), boiled at 95° C. for 5 min and loaded onto an SDS-PAGE gel (4% stacking gel, 10% separating gel). Then perform SDS-PAGE separation at a constant voltage of 90 V for about 110 minutes. At the end of electrophoresis, cut the separation gel and imaged with an Amersham Imager 600 (GE Healthcare) at 520 nm. The gel was then stained with Coomassie Brilliant Blue staining solution (0.1% Coomassie brilliant blue, 10% acetic acid, 50% methanol) for 30 min and destained with destaining solution (10% acetic acid, 30% methanol) to remove background color.

### 2. Results and Discussion

### 2.1 Optimization of Probe Labeling Conditions

To achieve optimal probe labeling, various labeling conditions were compared.

The fluorescence gel imaging showed that the fluorescence of labeled protein bands increased with increasing concentrations of the probe. The cell lysate can be effectively labeled at a probe concentration of 50 μM (FIG. 9a). Pre-incubate cell lysates with the probe or extend the “click” reaction time showed little influence on the labeling efficiency (FIG. 9b, 9d), while UV irradiation of 365 nm for 30 minutes increased the labeling efficiency (FIG. 9c).

The optimal conditions selected were incubation with 50 μM probe for 10 minutes, irradiation with 365 nm UV light for 30 minutes, and click reaction for 2 hours. For subsequent mass spectrometry experiments, a probe concentration of 100 μM could be used to enhance the signal intensity of labeled peptides.

### 2.2 Comparison of Different Probe Labeling

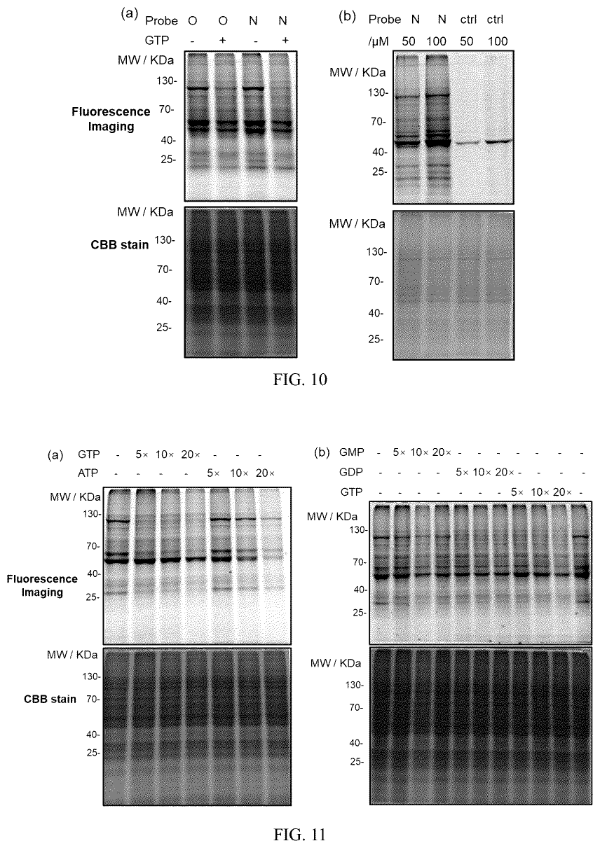

According to fluorescent gel imaging analysis, GTP-N probe exhibited stronger protein labeling compared to the GTP-O probe (FIG. 10a). The control probe without a photoreactive group could not form covalent bonds with proteins and labeled very few bands, demonstrating the necessity of photoaffinity labeling of the probes (FIG. 10b).

### 2.3 Competitive Probe Labeling Experiments

To verify the selectivity of probe labeling, competitive probe labeling experiments were conducted using ATP, GTP, GDP and GMP (FIG. 11). After incubating with each competitor on ice for 10 minutes, the probes were added for labeling. The fluorescence gel imaging showed that adding excess amount of GTP during probe labeling would significantly decrease the fluorescence intensity of several labeled proteins bands, whereas ATP or GMP showed non-specific competition, indicating good selectivity of this new type of GTP probes. Since many GTP-binding proteins also being able to bind GDP, using GDP as the competitor would show similar competitive bands as GTP.

### Example 3 Application of Photoaffinity Probes for Labeling GTP-Binding Proteins in Cell Lysates for Proteomic Analysis

Under optimized probe labeling conditions, HEK 293T cell lysates were labeled using GTP photoaffinity probes. High-resolution liquid chromatography-mass spectrometry (LC-MS/MS) was used to analyze the labeled proteins and peptides. The selectivity of probe labeling to GTP-binding proteins was validated based on the competitive effect of GTP on probe-labeled proteins.

### 1. Materials and Methods

### 1.1 Experimental Reagents

Ammonium bicarbonate and iodoacetamide (Bide), formic acid (Thermo Fisher), urea (Genview), biotin-PEG₃-azide and biotin-DADPS-azide (Leyan), trypsin and streptavidin (Thermo Fisher), Bradford protein assay kit (Beyotime). The remaining reagents are the same as in Section 1.1 of Example 2.

### 1.2 Experimental Equipment

Rotary shaker, C18 tip, EASY-nLC 1200 and Q-Exactive Orbitrap mass spectrometer (Thermo Fisher). The remaining equipment are the same as in Section 1.2 of Example 2.

### 1.3 Experimental Procedures

### 1.3.1 Preparation of Probe-Labeled HEK 293T Cell Lysate Proteomic Samples Via On-Beads Enzymatic Digestion

Under optimized conditions, HEK 293T cell lysates were labeled using the GTP photoaffinity probes. Peptide samples were prepared via the On-Beads enzymatic digestion method for proteomic study. Approximately 1 mg of proteins was labeled with 0.1 mM GTP probe and irradiated with 365-nm UV light for 30 min. To the solution were added freshly prepared tris-(2-carboxyethyl)phosphine (TCEP) until its concentration reached 1 mM, and a CuAAC reaction mixture to reach a final concentration of 1 mM CuSO₄, 0.1 mM tris[(1-benzyl-1H-1,2,3-triazol-4-yl)methyl]amine (TBTA), and 0.5 mM biotin-PEG₃-azide (for GTP-O probe labeled samples), or 0.5 mM biotin-DADPS-azide (for GTP-N probe labeled samples). The mixture was vortexed at room temperature for 2 hours and then precipitated in ice-cold methanol overnight.

The precipitated proteins were dissolved in 100 μL 4% SDS buffer (4% SDS, 150 mM NaCl, 50 mM HEPES, pH=7.4), and diluted with 900 μL SDS-free HEPES buffer (150 mM NaCl, 50 mM HEPES, pH=7.4). High-capacity streptavidin beads (50 μL per sample) were washed three times with 20 times volume of wash buffer (150 mM sodium chloride, 50 mM HEPES, pH=7.4) before adding to the samples. After incubation on a rotary shaker at room temperature for 2 hours, non-specifically bound proteins were washed away in a centrifugal filtration column using 1× PBS containing 1% SDS, 10× PBS, 1× PBS, water, and 50 mM ammonium bicarbonate solution with 8 M urea, each wash using 1 mL of solution for five times. The beads were transferred to a centrifuge tube using 50 mM ammonium bicarbonate solution containing 8 M urea and resuspended with 500 μL of this solution. The disulfide bonds were reduced by adding 20 mM TCEP and incubating at 37° C. for 1 hour, and then alkylated with 50 mM iodacetamide at room temperature in the dark for 30 minutes. After centrifugation at 3000 rpm for 2 minutes, the supernatant was gently removed. The beads were washed with 25 mM ammonium bicarbonate three times, resuspended in 100 μL 25 mM ammonium bicarbonate, and incubated with 1 μg of MS-grade trypsin (1 μg/μL) at 37° C. for 18 hrs.

The beads were washed with 200 μL of 25 mM ammonium bicarbonate solution, then centrifuged at 12000 rpm for 2 minutes to collect the supernatant. The procedure was repeated 6 times, and the supernatants were combined to obtain sample 1 (peptides without probe modification). For GTP-O probe labeled samples, the beads were resuspended in 200 μL of 70% acetonitrile solution containing 1% trifluoroacetic acid, vortexed for 5 minutes, then centrifuged at 12000 rpm for 2 minutes to collect the supernatant. The procedure was repeated 5 times, and the supernatants were combined to obtain sample 2 (containing probe-modified peptides). For GTP-N probe labeled samples, the beads were resuspended in 100 μL of 10% formic acid, incubated on a rotary shaker at room temperature for 30 minutes, then centrifuged at 12000 rpm for 2 minutes to collect the supernatant. The procedure was repeated 3 times, and the supernatants were combined. Trifluoroacetic acid was added to a final concentration of 1%, and incubated at 37° C. for 30 minutes to obtain sample 2. The peptide solutions were dried with a centrifugal concentrator and desalted using C18 tips, then frozen at −80° C. until LC-MS/MS detection. Samples were dissolved in 20 μL of pure water, 4 μL of sample 1 and 8 μL of sample 2 were injected for analysis.

### 1.3.2 LC-MS/MS Detection Method and Data Analysis

An EASY-nLC 1200 liquid chromatography coupled with a Q-Exactive Orbitrap mass spectrometer was employed for LC-MS/MS analysis. A homemade C18 capillary column (inner diameter 75 μm, column length 18 cm) packed with Reprosil-Pur120 C18-AQ (Dr. maisch, particle size 3 m) was utilized as the separation column. The precolumn (inner diameter 150 μm, column length 4 cm) was packed with Reprosil-Pur120 C18-AQ (Dr. maisch, particle size 5 μm,). The mobile phase A was 0.1% formic acid, while mobile phase B was 0.1% formic acid in 80% acetonitrile, and the flow rate was 300 nL/min. The gradient elution condition was as follows: 0-5 min, 5-10% B; 5-140 min, 10-35% B; 140-145 min, 35-55% B; 145-146 min, 55-88% B; 146-156 min, 88-99% B; 156-181 min, 99% B.

The Q-Exactive mass spectrometer was operated in positive ion mode with a spray voltage of 2.1 kV and ion transfer tube temperature of 275° C. The m z range for full MS acquisition was 350-1800. Top 20 ions were fragmented and scanned to obtain tandem MS spectra. For full MS scans, the resolution was 70000, with a dynamic gain control of 1×10⁶ ions, and a maximum injection time of 50 ms. For tandem MS scans, the resolution was 17500, with a dynamic gain control of 5×10⁴ ions and a maximum injection time of 50 ms. Isolation width was set to 2 m/z, and collision energy was standardized at 28, 29, and 30.

The obtained MS raw files were analyzed using MaxQuant (v2.1.4.0) software, and the search was performed against the UniProt human protein database (as of November 2022, with a total of 20,401 proteins). Enzyme specificity was set to trypsin, cysteine carbamidomethylation was set as a fixed modification, methionine oxidation and N-terminal acetylation were set as variable modifications. To identify probe-labeled sites with the GTP-N probe, modification after releasing GTP and biotin (+252.1950 Da) was set as a variable modification for amino acids YECDHKS that showed labeling preference by diazirine.

### 2. Results and Discussion

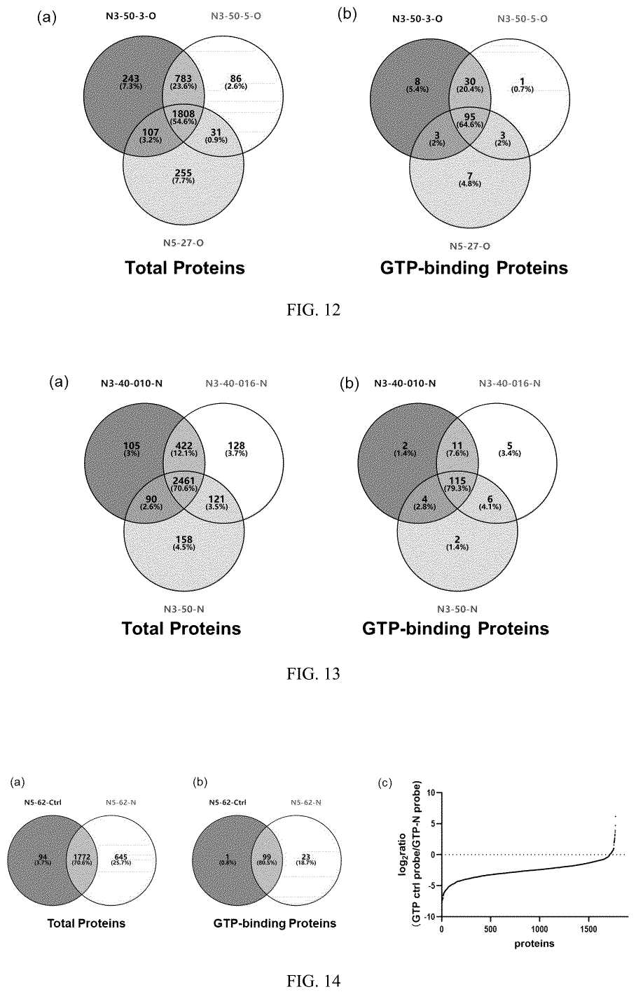

2.1 Proteomic Analysis of HEK 293T Cell Lysate Labeled with GTP Probes For the GTP-O probe, a total of 1808 proteins were identified in three biological replicates, including 95 reported GTP-binding proteins (FIG. 12a and FIG. 12b).

For the GTP-N probe, a total of 2461 proteins were identified in three biological replicates, including 115 reported GTP-binding proteins (FIG. 13a and FIG. 13b), indicating slightly better labeling efficiency compared to the GTP-O probe.

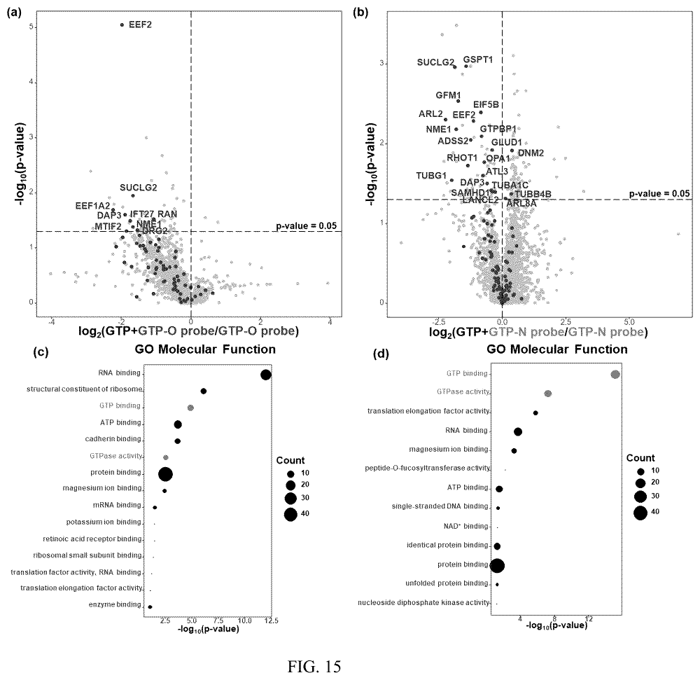

For the control probe without the photoreactive group, fewer proteins were identified compared to the GTP-N probe, and almost all GTP-binding proteins identified with the control probe were also identified with the GTP-N probe (FIG. 14a and FIG. 14b). Although over 2000 total proteins and 100 GTP-binding proteins were identified with the control probe, the protein intensities detected with the control probe were much lower than that of the GTP-N probe (FIG. 14c). This suggested that covalent connection with the photoreactive group would help capture target proteins with a much higher efficiency.

### 2.2 Effect of GTP Competition on GTP-O or GTP-N Probe Labeling

With the presence of GTP as the competitor, labeling of GTP-binding proteins would be hindered. Adding 10 times probe concentration of GTP during probe labeling, compared with the probe-labeling group without competitors, more specific GTP-binding proteins could be identified using label-free quantification (LFQ) method. The competition experiment and LFQ analysis were conducted in three biological replicates (FIG. 15a and FIG. 15b). According to volcano plot analysis, the labeling efficiency of most GTP-binding proteins decreased after adding GTP competition. The competition results showed that the GTP-N probe exhibited better selectivity than the GTP-O probe for annotated GTP-binding proteins. The gene ontology (GO) molecular functional (MF) analysis of proteins with significantly lower intensities in the GTP competition group also showed better enrichment of GTP-binding and GTPases activity related proteins with the GTP-N probe (FIG. 15c and FIG. 15d).

2.3 Identification of Probe-Modified Sites on Labeled Proteins

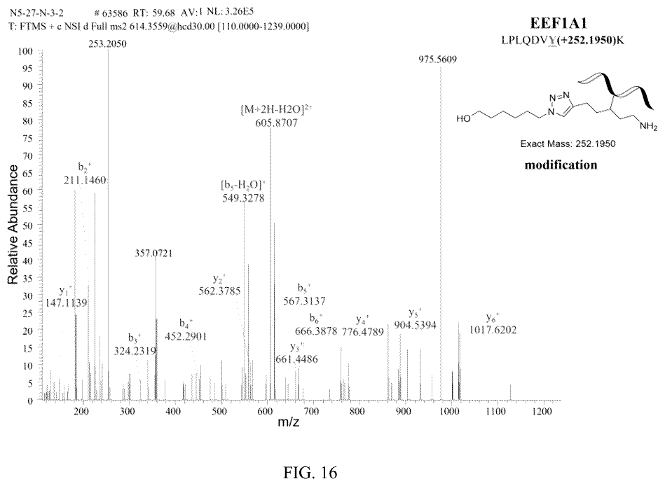

For samples labeled with the GTP-O probe, probe-modified sites were not detected. For MS samples labeled with the GTP-N probe, the click reactions were performed using biotin-DADPS-azide with acid-cleavable silicon-oxygen bonds. After tryptic digestion, peptides without probe modification would be dissolved in the supernatant and were collected using 25 mM ammonium bicarbonate solution (sample 1). At this stage, peptides containing probe modifications remained tightly bound to the beads due to the presence of biotin groups. The beads were then incubated with 10% formic acid to break the silicon-oxygen bonds on the biotin group, allowing these peptides to dissociate from the beads. The mixture was then supplemented with 1% trifluoroacetic acid and incubated at 37° C. for 30 minutes to release GTP. The resulting supernatant containing probe-modified peptides were collected (sample 2). Using this method, several modified peptides of GTP-binding proteins were identified, such as EEF1A1. The b-ions and y-ions in the tandem MS spectra of these modified peptides were matched with theoretical values, confirming the peptide sequences and modifications (FIG. 16).

### Example 4: Application of Photoaffinity Probe Labeling for the Analysis of G Protein Inhibitors

The GTP-N probe labeling was applied in the identification of target proteins of the GTP competitive inhibitor EHT 1864 in HEK 293T cell lysates. Through quantitative analysis of decreased probe labeling efficiency with increasing concentrations of EHT 1864, potential target proteins of EHT 1864 were analyzed.

### 1.1 Experimental Reagents

EHT 1864 (Bide, CAS: 754240-09-0), other reagents were the same as those in Section 1.1 of Example 3.

### 1.2 Experimental Equipment

Same as those in Section 1.2 of Example 3.

### 1.3 Experimental Procedures

### Preparation of Proteomic Samples for G Protein Inhibitor Competitive Probe Labeling of HEK 293T Cell Lysates

Using the On-Beads digestion method, the GTP-N probe was used for protein labeling of HEK 293T cell lysates. For the competition experiments, a GTP-competitive inhibitor EHT 1864 was added before probe labeling to inhibit probe binding with targeted GTP-binding proteins. The final concentration of the probe was 20 μM, and EHT 1864 was used at concentrations of 20 μM, 40 μM, 100 μM, and 200 μM, respectively. The inhibitor was pre-incubated with cell lysates on ice for 10 minutes, followed by the GTP-N probe labeling, sample preparation, and LC-MS/MS analysis, same as in Section 1.3 of Example 3.

### 2. Results and Discussion

EHT 1864 is a GTP-competitive inhibitor that directly targets Rac family proteins at their GTP-binding sites, reducing cell viability and disrupting cytoskeletal formation and cell adhesion in various cancer cells. By using the GTP-N probe labeling with increasing concentrations of EHT 1864 in HEK 293T cell lysates, potential target proteins of EHT 1864 could be identified through quantitative analysis. Analyzing the influence of EHT 1864 on the labeling efficiency of the GTP-N probe would help with mechanistic studies of this inhibitor.

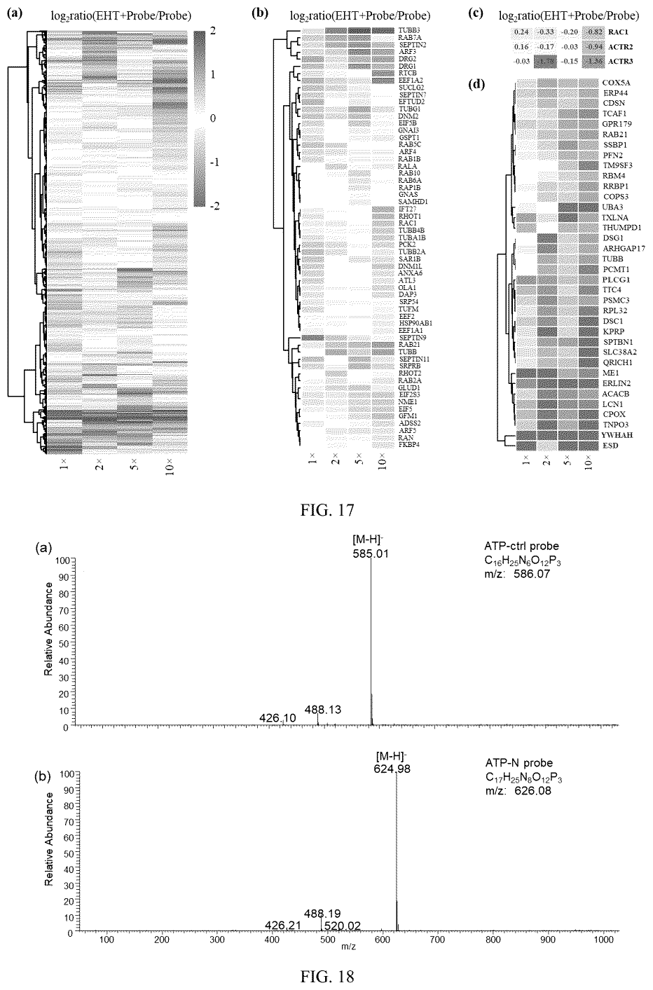

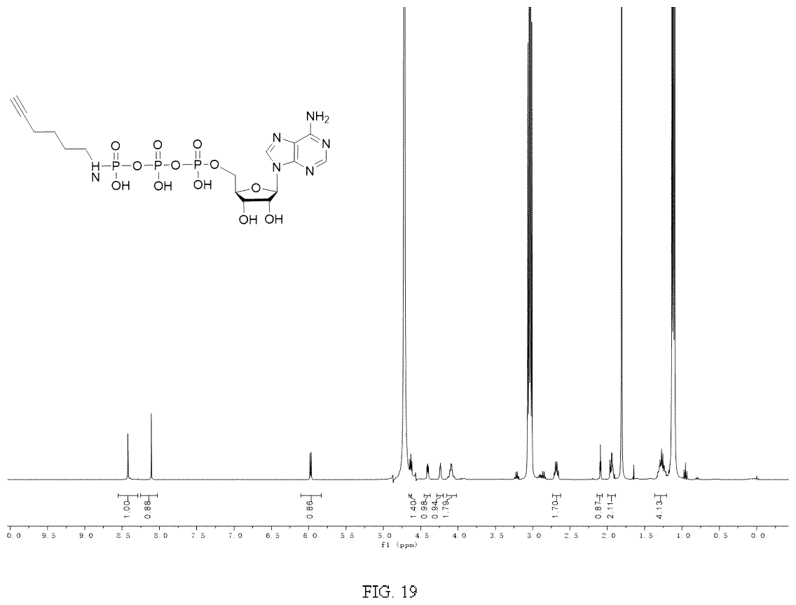

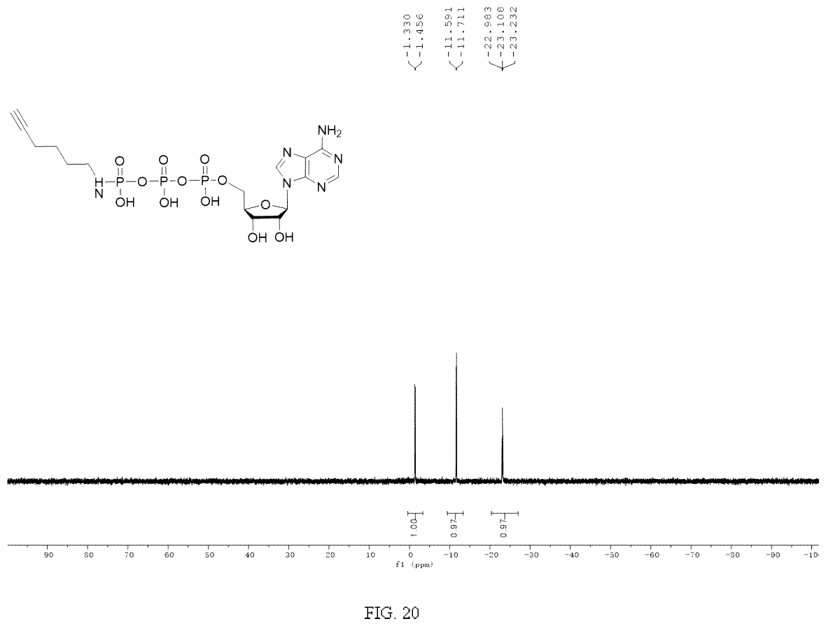

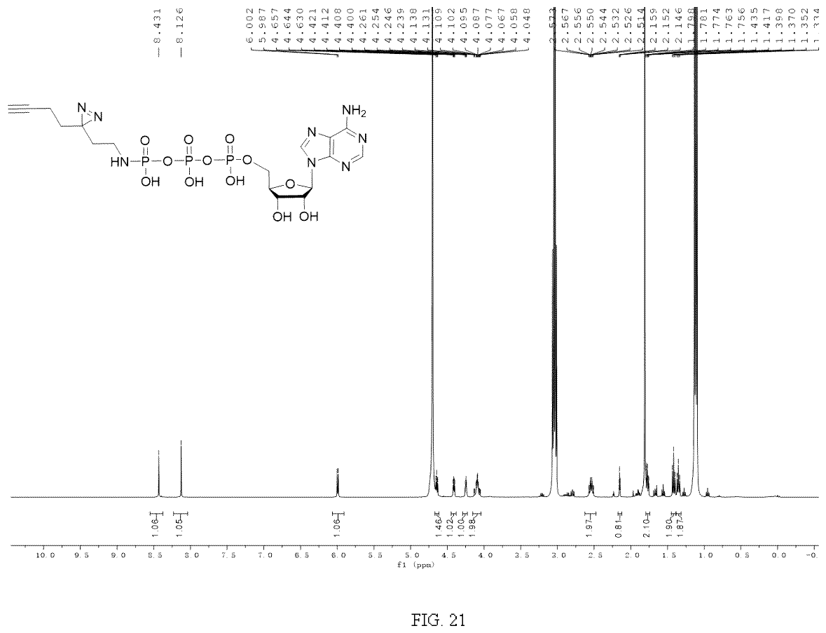

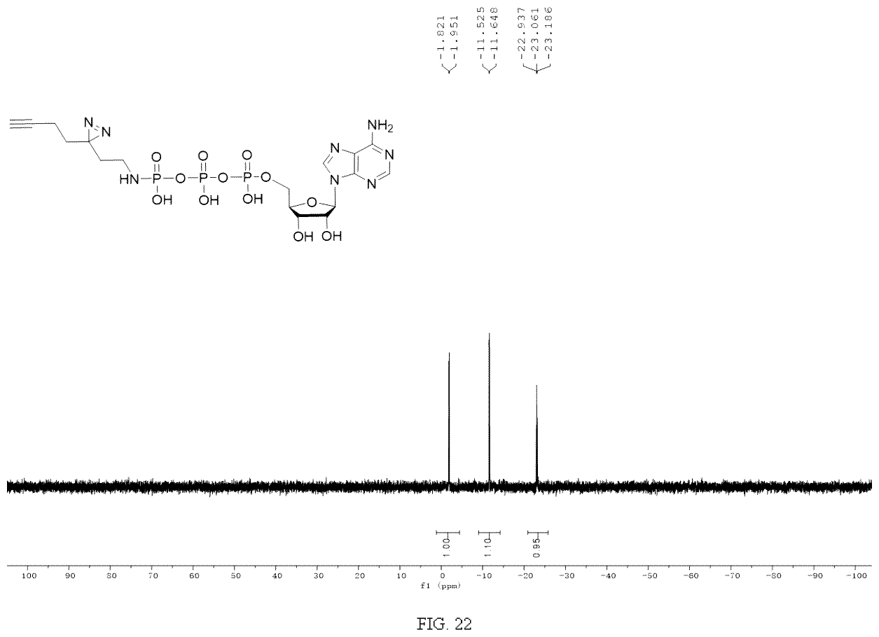

Several proteins, including GTP-binding proteins, showed decreased intensity with increasing inhibitor concentrations, suggesting that they could be potential inhibitor targets (FIG. 17). Identified proteins were screening with the following four criteria: (1) at 10× probe concentration of inhibitor, competition group/probe group<0.5, i.e., log₂(competition group/probe group)<−1; (2) at 5× probe concentration of inhibitor, competition group/probe group<⅔, i.e., log₂(competition group/probe group)<−0.58; (3) at 2× probe concentration of inhibitor, competition group/probe group<1.2, i.e., log₂(competition group/probe group)<−0.26; (4) at 1× probe concentration of inhibitor, competition group/probe group<1.2, i.e., log₂(competition group/probe group)<−0.26. The screened proteins are involved in biological processes such as cell DNA and centrosome replication and regulation, translation, protein transport, and osteoblast differentiation. Their molecular functions related with DNA replication, translation, cell cycle regulation, and cell adhesion, etc. The reduced cell viability and disruption of cancer cell cytoskeletal formation and cell adhesion by EHT 1864 treatment may be related to its impact on these proteins. EHT 1864 may directly or indirectly bind to these proteins or affect their upstream pathways, thereby influencing protein activity.

When incubating with gradient concentrations of EHT 1864 before probe labeling, the mass spectrometry signal for its target protein RAC1 showed a decreasing trend, but the overall reduction was not significant. This might be due to insufficient competition of the inhibitor or the accuracy of the mass spectrometry quantification needing further improvement (FIG. 17c). Additionally, some proteins previously reported to be potentially affected by EHT 1864 were identified, including ACTR2, ACTR3, and PLCG1, with overall decreased protein intensities detected in the competition groups using EHT 1864.

### Example 5 Design and Synthesis of a Novel ATP-N Photoaffinity Probe

### 1.1 Experimental Reagents

Adenosine-5′-triphosphate disodium salt hydrate (ATP) (Sigma-Aldrich), other reagents were the same as those in Section 1.1 of Example 1.

### 1.2 Experimental Equipment

Same as in Section 1.2 of Example 1.

### 1.3 Experimental Procedures

### 1.3.1 Synthesis and Purification of the Probes

### ATP-Ctrl Robe:

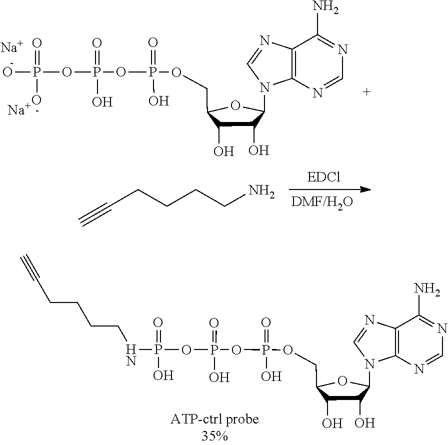

Except for replacing the GTP disodium salt with adenosine 5′-triphosphate disodium salt hydrate (0.2 mmol, 0.1102 g), all other steps were consistent with the synthesis of the GTP-N control probe (in Section 1.3 of Example 1). The liquid-phase purification conditions were the same as those for the GTP-N probe. Gradient elution conditions: 0-15 min, 0-74.5% B; 15-20 min, 74.5% B; 20-25 min, 74.5-100% B; 25-30 min, 100% B; 30-33 min, 100-10% B; 33-36 min, 10% B. The ATP-ctrl probe was eluted at 16-18 minutes, probe-containing eluents were combined and lyophilized to yield a white solid. The purification was repeated until a pure product was obtained, with a yield of approximately 34%. The probe concentration was determined by UV absorption at 259 nm (extinction coefficient ε=15400 M−1 cm−1). The probe was aliquoted and stored at −80° C. The structure and purity of the probe were confirmed with HRMS and NMR. ¹H NMR (400 MHz, D₂O) δ 8.42 (s, 1H), 8.11 (s, 1H), 5.97 (d, J=6.0 Hz, 1H), 4.63 (t, J=5.5 Hz, 1H), 4.41 (dd, J=5.1, 3.4 Hz, 1H), 4.24 (p, J=2.9 Hz, 1H), 4.15-4.03 (m, 2H), 2.73-2.63 (m, 2H), 2.09 (t, J=2.6 Hz, 1H), 1.99-1.91 (m, 3H), 1.35-1.20 (m, 4H). ³¹P NMR (162 MHz, D₂O) δ-1.39 (d, J=20.5 Hz), −11.65 (d, J=19.5 Hz), −23.11 (t, J=20.2 Hz). HRMS (ESI) m/z calcd. for C₁₆H₁₅N₆O₁₂P₃[M-H]⁻: 585.0665, found: 585.0629.

### Atp-N Probe:

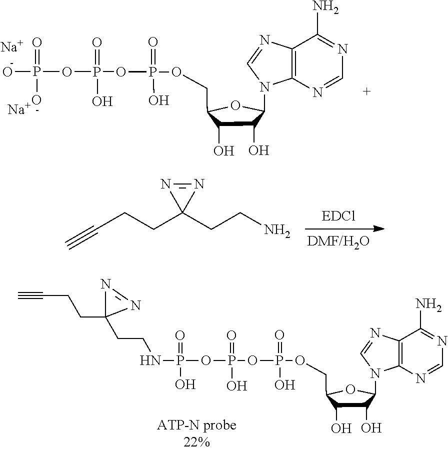

Except for replacing 5-hexyn-1-amine (0.22 mmol, 21.4 mg) with 3-aminoethyl-3-(but-3-ynyl)diazirine (0.22 mmol, 30.1 mg), all other steps were the same as those for the synthesis and purification of the ATP-N control probe. The ATP-N probe was eluted at 23-25 minutes, probe-containing eluents were combined and lyophilized to yield a white solid with a yield of 22%. The probe concentration was determined by UV absorption at 259 nm (extinction coefficient ε=15400 M⁻¹ cm⁻¹). The probe was aliquoted and stored at −80° C. The structure and purity of the probe were confirmed with HRMS and NMR. ¹H NMR (400 MHz, D₂O) δ 8.43 (s, 1H), 8.13 (s, 1H), 5.99 (d, J=5.9 Hz, 1H), 4.64 (t, J=5.5 Hz, 1H), 4.41 (dd, J=5.1, 3.5 Hz, 1H), 4.25 (q, J=3.0 Hz, 1H), 4.16-4.03 (m, 2H), 2.62-2.46 (m, 2H), 2.15 (t, J=2.7 Hz, 1H), 1.77 (dd, J=7.2, 2.6 Hz, 2H), 1.42 (t, J=7.3 Hz, 2H), 1.35 (t, J=7.3 Hz, 2H). ³¹P NMR (162 MHz, D₂O) δ-1.89 (d, J=21.0 Hz), −11.59 (d, J=19.9 Hz), −23.06 (t, J=20.2 Hz). HRMS (ESI) m/z calcd. for C₁₇H₂₅N₈O₁₂P₃[M-H]⁻: 625.0727, found: 625.0691.

### Example 6 Analysis of ATP-N Probe Labeling of Cell Lysates with In-Gel Fluorescence Imaging

The labeling efficiency of the ATP-N probe was analyzed with in-gel fluorescence imaging. To verify whether this ATP-N probe can effectively label ATP-binding proteins, competition experiments with ATP, ADP, and AMP were conducted to validate the selectivity of the probe towards ATP-binding proteins.

### 1.1 Experimental Reagents

Same as in Section 1.1 of Example 2.

### 1.2 Experimental Equipment

Same as in Section 1.2 of Example 2.

### 1.3 Experimental Procedures

HEK 293T cell lysates were labeled with the ATP-N probe. Before labeling, 0.5 mM EDTA, 1 mM magnesium chloride, and 1 mM calcium chloride were added to the lysates. After UV irradiation, proteins were precipitated in a solution of chloroform: methanol: water (4:3:1, v/v) for three times. The precipitated proteins were dissolved in 10 μL of 4% SDS buffer (4% SDS, 50 mM HEPES, 150 mM NaCl, pH=7.4), and diluted to 100 μL with the SDS-free buffer (50 mM HEPES, 150 mM NaCl, pH=7.4) before proceeding to the click reaction. The remaining steps were consistent with those in Section 1.3 of Example 2.

### 2. Results and Discussion

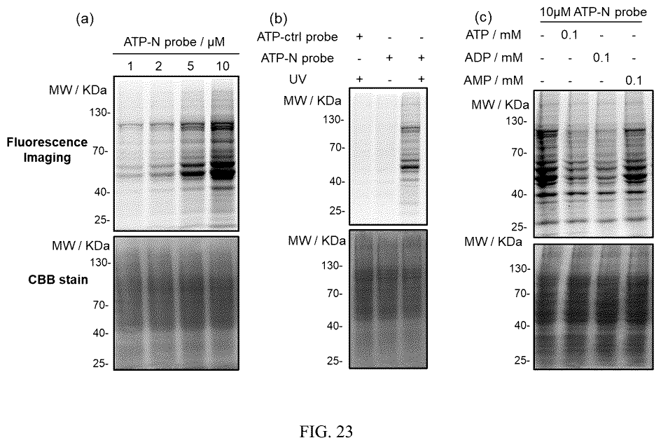

As shown in FIG. 23a, the ATP-N probe effectively labeled cell lysates, showing clear fluorescent bands even at concentrations as low as 1 μM. To ensure a good response for subsequent mass spectrometry sample analysis, a probe concentration of 10 μM was selected for labeling. For the control probe without photoreactive group, no fluorescent bands were visible, similar to the ATP-N probe without UV irradiation (FIG. 23b), suggesting the importance of photoreactive group for probe labeling.

To verify the selectivity of the probe labeling, competition experiments were conducted using ATP, ADP, and AMP. The fluorescence gel imaging results (FIG. 23c) showed similar specific competition bands when ATP and ADP were used as competitors, possibly because most ATP-binding proteins can also bind to ADP. The competition effect of AMP was not significant, indicating good selectivity of the probe.

### Example 7: Proteomic Analysis of Cell Lysates Labeled with the ATP-N Probe

Using the labeling conditions mentioned above, HEK 293T cell lysates were labeled with the ATP-N probe. Labeled proteins were prepared into proteomic samples and analyzed with high-resolution LC-MS/MS. To verify the selectivity of the ATP-N probe labeling towards ATP-binding proteins, competition experiments with ATP were performed.

### 1.1 Experimental Reagents

Same as in Section 1.1 of Example 3.

### 1.2 Experimental Equipment

Same as in Section 1.2 of Example 3.

### 1.3 Experimental Procedures

### 1.3.1 Preparation of Mass Spectrometry Samples

1 mg of cell lysate was labeled with 10 μM ATP-N probe. The subsequent steps were the same as in Section 1.3 of Example 3.

### 1.3.2 LC-MS/MS Detection Method

The C18 pre-column (Thermo Fisher, 164564-CMD, 100 μm×2 cm) and analytical column (Thermo Fisher, 164941, 75 μm×25 cm) used in the LC-MS/MS analysis were commercially available. The liquid chromatography (Vanquish Neo, Thermo Fisher) settings were as follows: Solvent A was 98% H2O with 0.1% FA, and Solvent B was 98% ACN with 0.1% FA. The flow rate was 300 nL/min, with the elution gradient as follows: 0 minutes, 2% B; 5 minutes, 26% B; 127 minutes, 36% B; 137 minutes, 72% B; 142 minutes, 80% B; and 143-153 minutes, 80% B. The mass spectrometer (Orbitrap Exploris 480, Thermo Fisher) settings were: full MS scan in positive ion mode, electrospray ion source voltage 1.9 kV, ion transfer tube temperature 320° C., scan m/z range 350-1500, resolution of 120000, AGC target 300%, maximum injection time 25 ms. For dd-MS², resolution was 15000, AGC target 50%, maximum injection time was automatically selected, and the cycle time for MS scanning was set to 3 s. DDA mode was used for data acquisition and the data collection time was 0-142 minutes.

### 1.3.3 Data Processing Method

The obtained MS raw files were analyzed using Proteome Discoverer 2.5 software, and searched against the Uniprot human protein database (as of November 2022, total proteins: 20401). Enzyme specificity was set to trypsin, cysteine carbamidomethylation was set as a fixed modification, and methionine oxidation and N-terminal acetylation were set as variable modifications. To identify probe-labeled sites with the ATP-N probe, modification after releasing GTP and biotin (+252.1950 Da) was set as a variable modification for amino acids CDEHKRSTY that showed labeling preference by diazirine.

### 2. Results and Discussion

HEK 293T cell lysates were labeled with 10 μM ATP-N probe. Labeled proteins were enriched with streptavidin beads, and digested with trypsin to prepare peptide samples for LC-MS/MS analysis. Adding 100 μM ATP as the competitor during probe labeling, the labeling of ATP-binding proteins would be interfered. Compared with the probe-labeling group without competitors, more specific ATP-binding proteins could be identified with label-free quantitative (LFQ) analysis.

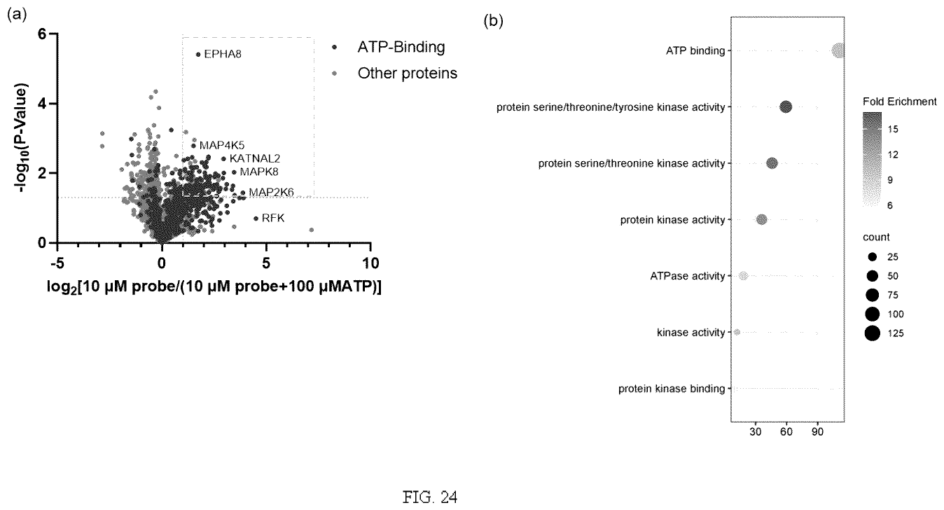

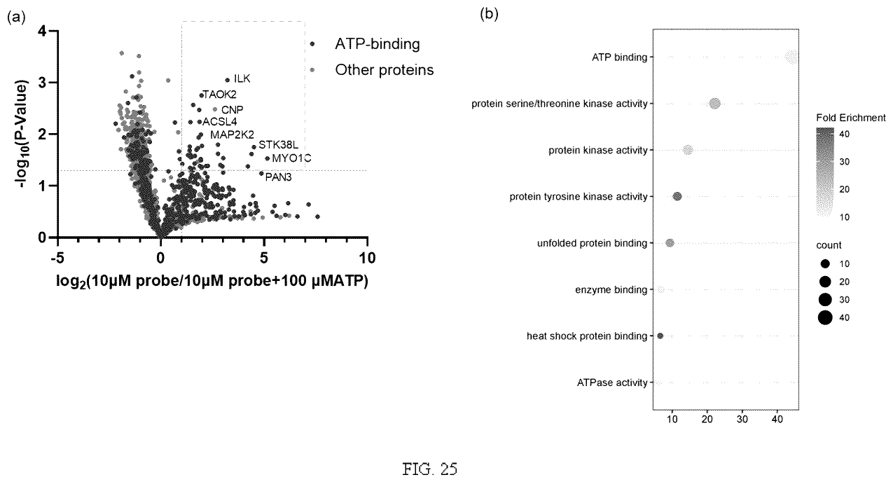

As shown in the Volcano plot (FIG. 24a), the normalized abundance of most known ATP-binding proteins significantly decreased after adding ATP as a competitor. The competition experiment and LFQ analysis were conducted in three biological replicates. More than 160 proteins showed significantly decreased protein intensity in the competition group in all replicates (ratio>2, p-value<0.05). The gene ontology molecular functional (GO-MF) analysis of these proteins revealed that approximately 129 proteins were previously annotated ATP-binding proteins, accounting for over 80% of all screened proteins, and around 78 of them were kinases, indicating good enrichment of kinases using this probe.

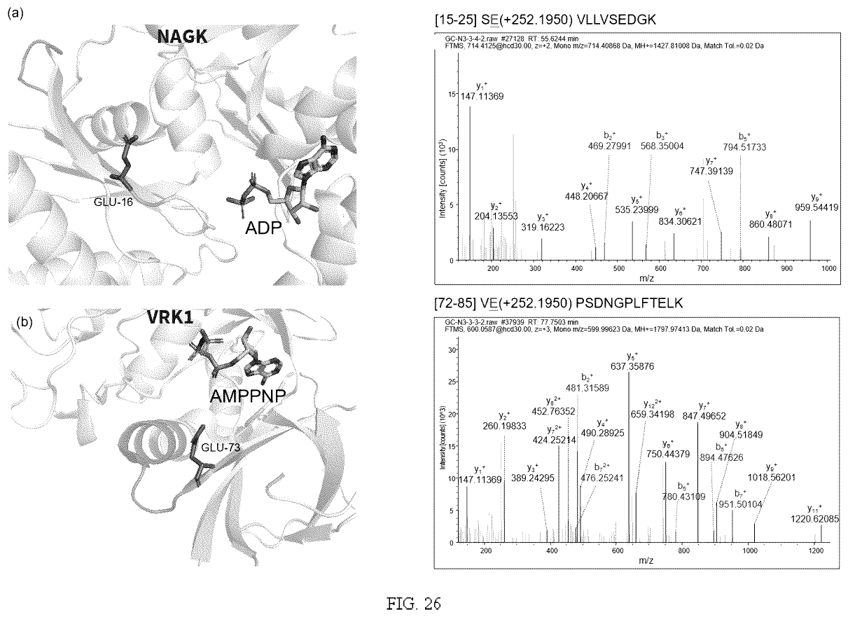

Quantitative analysis of probe-modified peptides was also conducted. About 44 proteins showed significantly decreased protein intensity in the competition group in all three replicates (ratio>2, p-value<0.05). Except for CNP, all the other identified proteins are annotated ATP-binding proteins. Although CNP was not previously known to bind ATP, its function is related to nucleoside phosphate. The Volcano plot showed the results of LFQ analysis (FIG. 25a), and GO-MF analysis was performed on the screened proteins (FIG. 25b). Most screened proteins were related to ATP binding and kinase activity, indicating good labeling effect and selectivity of the probe for ATP-binding proteins, as well as its ability to identify their probe binding sites, facilitating the analysis of ATP-binding protein binding pockets and activities.

Based on the analysis of modified peptide, this probe can facilitate identification of ATP-binding sites in proteins. After cleavage under acidic conditions, the probe modification added a mass increment of 252.1950 Da to peptides, allowing high-resolution LC-MS/MS analysis to identify probe-modified amino acids. For instance, in the samples of HEK 293T cell lysates labeled with the ATP-N probe, N-acetylglucosamine kinase (NAGK) showed modification near its ATP-binding pocket on Glu16 (FIG. 26a). Another example is VRK1, a nuclear chromatin kinase overexpressed in many tumor types and associated with poor prognosis. From a reported crystal structure, the probe labeled Glu73 residue is close to VRK1's ATP-binding pocket (FIG. 26b). These examples demonstrated that the ATP probe developed in this study can not only be used to analyze proteins enriched by the probe, but also determine the probe-modified sites, which usually located near the ATP-binding pockets of proteins. This method could be further applied in the research regarding functions, activities and inhibitors of ATP-binding proteins.

### Example 8: Analysis of Potential Targets of Kinase Inhibitors Based on ATP Photoaffinity Probe Labeling

Considering the high labeling efficiency and selectivity of the probe for kinases, further analysis was conducted using the probe to explore potential targets of kinase inhibitors. Staurosporine (STS) is a widely studied broad-spectrum kinase inhibitor with strong affinities for the ATP-binding sites of various kinases, making it a typical ATP-competitive kinase inhibitor. Therefore, STS was chosen as an example to study the feasibility of using the probe to analyze potential targets of kinase inhibitors.

### 1.1 Experimental Reagents and Equipment

Same as in Section 1.1 and 1.2 of Example 7.

### 1.2 Experimental Procedures

The preparation of mass spectrometry samples was the same as in Section 1.3 of Example 7, except that ATP was replaced with different concentrations of staurosporine.

### 2. Results and Discussion

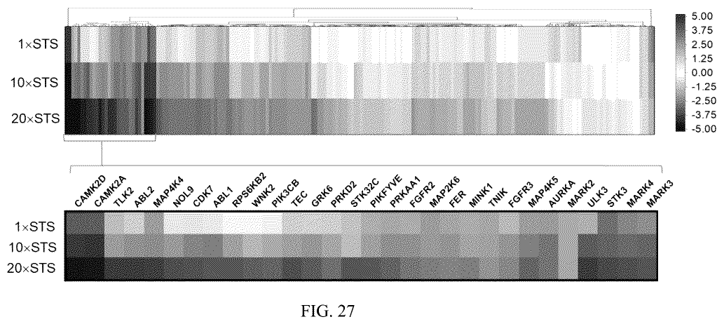

Prior to probe labeling, cell lysates were pre-incubated on ice with staurosporine at 1×, 10×, and 20× concentration of the ATP-N probe, and compared with the control group without staurosporine treatment. Potential target proteins of kinase inhibitors were screened based on the abundance ratio between the competition group and the control group through LC-MS/MS analysis. Theoretically, as the concentration of the inhibitor increases, the labeling efficiency of its target proteins should gradually decrease. Therefore, proteins were screened based on their abundance ratios (10×STS/1×STS>1 and 20×STS/10×STS>1), identifying proteins whose detected abundance decreased with increasing inhibitor concentrations. Proteins with lower confidence were filtered out, leaving proteins that met the criteria for further analysis. Most proteins with reduced labeling efficiency after staurosporine treatment were ATP-binding proteins, including kinases, and this trend increased with higher inhibitor concentrations. Among proteins with significantly reduced labeling in the competition group, several known staurosporine targets were identified (FIG. 27). Since the data presented in this example represent only one biological replicate, there may be some errors. Typically, using the average values and p-values from three biological replicates would yield more reliable results.

It should be noted that the above examples are merely for illustrating the technical solutions of the present invention and are not intended to limit them. Although the present invention has been described in detail with reference to the given examples, those skilled in the art may make modifications or equivalent replacements to the technical solutions of the present invention as needed without departing from the spirit and scope of the technical solutions of the present invention.

1. A nucleoside triphosphate photoaffinity probe, having a structural formula as shown in formula I: 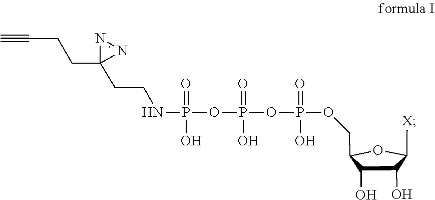
wherein X is selected from

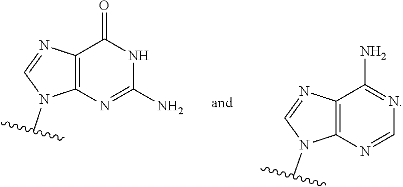 [SMILES: *n1cnc2c(=O)[nH]c(N)nc21 ; *n1cnc2c(N)ncnc21]

2. A method for preparing the nucleoside triphosphate photoaffinity probe according to claim 1, comprising a synthetic route as follows: 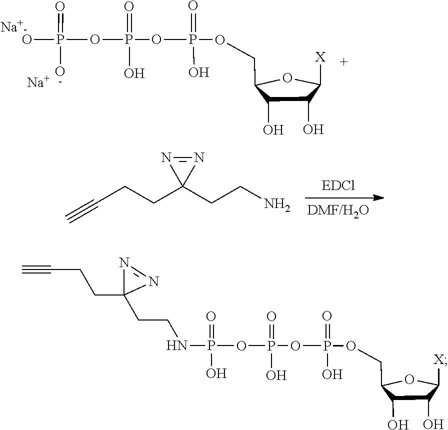
wherein X is selected from

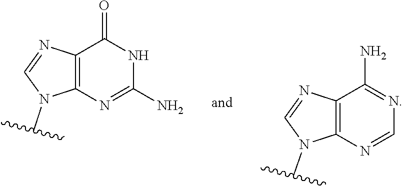 [SMILES: *n1cnc2c(=O)[nH]c(N)nc21 ; *n1cnc2c(N)ncnc21]

3. A detection kit, comprising at least the nucleoside triphosphate photoaffinity probe according to claim 1.

4. The detection kit according to claim 3, further comprising any one or more of the following reagents: reaction enhancers, enzyme reagents, buffers, and cleaning solutions.

5. An application of the nucleoside triphosphate photoaffinity probe according to claim 1 in the research related to GTP/ATP-binding proteins.

6. The application according to claim 5, wherein the research related to GTP/ATP-binding proteins at least comprises:
(a) proteomic MS analysis of GTP/ATP-binding proteins in cell lysates; and
(b) analysis of G-protein inhibitors or kinase inhibitors.

7. The application according to claim 6, wherein a specific method for application (a) comprises: adding the nucleoside triphosphate photoaffinity probe to HEK 293T cell lysates, followed by UV irradiation, and performing click chemistry with biotin-DADPS-azide.

8. The application according to claim 7, wherein the specific method for application (a) further comprises: using LC-MS/MS to detect and analyze the proteins labeled by the nucleoside triphosphate photoaffinity probe, assigning peptide segments from the obtained mass spectrometry data using MaxQuant software or Proteome Discoverer 2.5, and performing GO analysis using DAVID website.

9. The application according to claim 6, wherein in application (b), the G-protein inhibitor is EHT 1864, and the kinase inhibitor is staurosporine.

10. The application according to claim 6, wherein the application (b) comprises analysis and identification of potential target proteins of G-protein inhibitors or kinase inhibitors.
